(19) United States
(12) Patent Application Publication
(10) Pub. No.: US 2004/0039390 A1
(43) Pub. Date: Feb. 26, 2004

(54) Hollow guide rod for orthopaedic surgery

(76) Inventor: John R. Pepper, Cheshire, CT (US)

Correspondence Address: JOHN R. PEPPER, 224 BEACON HILL DRIVE, CHESHIRE, CT, 06410, US

(21) Appl. No.: 10/643,979

(22) Filed: Aug. 20, 2003

Related U.S. Application Data
(60) Provisional application No. 60/404,627, filed on Aug. 20, 2002.
(60) Provisional application No. 60/414,890, filed on Oct. 1, 2002.

(51) Int. Cl. A61B 17/16

(52) U.S. Cl. 606/79

(57) ABSTRACT

A hollow guide rod for long bone surgery provides a pathway for fluids, as well as a pathway for information transmission of positional information from a transducer. An enlarged end will stop reamers from disengaging. High canal pressures are vented through the hollow channel, reducing the risk of pulmonary embolism.

### CROSS REFERENCE TO RELATED APPLICATION

This application claims the benefits of Provisional Patent Application Ser. No. 60/404,627 filed Aug. 20, 2002 and Provisional Patent Application Ser. No. 60/414,890 filed Oct. 1, 2002.

### FEDERALLY SPONSORED RESEARCH

This was not federally sponsored research.

### BACKGROUND OF THE INVENTION

Fracture fixation at the present time is done by surgically inserting a rod down the marrow canal of broken long bones. The rod allows rapid return to normal life and much improved healing times.

To fix a fracture, the bone must be realigned. High energy trauma, such as motor vehicle accidents, often results in displaced fractures where one end of the fragment is not aligned with the other. The passing of device through the bone is needed, so the fracture must be aligned, or reduced. This is frequently done with the use of a guide rod. The guide rod is small (two to three millimeter) diameter solid piece of wire. It is inserted into the center of one of the bone fragments, usually at a place that requires minimal soft tissue dissection. The tip of the rod may be prebent to allow selectable angle steering of the rod, in an attempt to cross the gap. Intra-operative fluoroscopy, (x ray) is used to determine the position of the rod in relation to the bone fragments. The fluoroscope, or C arm is a device with an X ray source located on a pod, and about four feet away is a receiver. The C arm is on a large cart that is cumbersome to position. Frequent rod advances, and fluoroscopy checks are required to find and go through the loose fragment. Verification is done by a perpendicular image to verify the rod is in the canal. A comminuted (multi-piece) fracture could require repeating this process for each fragment. This is one of the more tedious portions of the procedure, requiring much more skill than would thought to be required. Muscle contraction, swelling, leaking and slippery bone marrow (fat) add to the challenge. There is considerable exposure to radiation with this technique. Often, the speed at which a case can be done depends not only on the surgeon's skill but on that of the fluoroscope operator. The technician must make adjustments to maintain image location, angle, ability to cross check the progress in the case. The communication between x-ray technician and surgeon is critical to get the positional information to the surgeon. It can be very stressful. Delays in the reduction of the fracture can have dire consequences for the patient who is gravely injured and can not tolerate the rigors of surgery as would a healthy patient,. Often the patient will have head or serious chest injuries. Making the reduction fast is critical.

Once the fracture is reduced, reaming is the next step. Intramedullary canal reaming is performed to prepare a long bone for hip arthroplasty or fracture fixation devices. The irregular shape and changing cross section of the bone must be made uniform to fit the implant devices. The usual technique is to attach a cylindrical reamer to a flexible shaft and use a power drill to cut away bone from the inside out. Several passes are required to open the canal up in many cases, as each cut takes off only one half to one millimeter of bone. A guide rod acts as a path for the reamers to follow and a means to retrieve broken reamer components. Guide rods typically have enlarged ends to prevent reamers from going over the end and becoming dissociated deep within the bone where retrieval is very difficult.

Early reamers had large cross sections to center the cutter in the previous cutters path. These reamers formed pressure plugs and would raise the pressure in the canal ahead of the reamer. The marrow fat would be forced out of the canal into the venous system. In some cases the fat will create problems in the lungs, called pulmonary embolism. It has been a fatal condition in a number of cases.

Device manufacturers, upon surgeon's request have modified the reamers to allow less pressure build up in front of the cutting area. The result is lower pressure, but the study of this problems illustrates the need to take in consideration all design facets that could reduce risk to the patient. Often the flutes of the reamer will clog with debris, especially on the first pass where the flutes must eject not only the bone chips but the entire contents of the marrow canal. It is a messy part of the procedure.

To avoid risks associated with pulmonary embolisms, some doctors prefer to insert a rod with out reaming the canal. The rods are smaller and will fit down the natural irregularly shaped canal. The risk of pulmonary embolism is this different technique still exists, and perhaps even to a greater extend. The rod is hammered into the canal, and acts as a piston, compressing the material and fat in the canal ahead of it. The egress of the fat can be in the small spaces between the rod and the canal and back up the path of the bone, thorough the center of the nail, or into the venous system.

While rodding is much preferred to earlier techniques such as traction, clearly there are risks associated with the surgery. Radiation exposure, pulmonary embolism are two conditions that can harm compromised patients. This invention reduces the risk to the patients.

### BREIF SUMMARY OF THE INVENTION

A hollow guide rod allows fluid in and out of the bone canal, and allows a pathway for transmission means for positional information.

### BRIEF DESCRIPTION OF THE DRAWINGS

FIG. 1, Hollow Guide rod with ball end-cross section

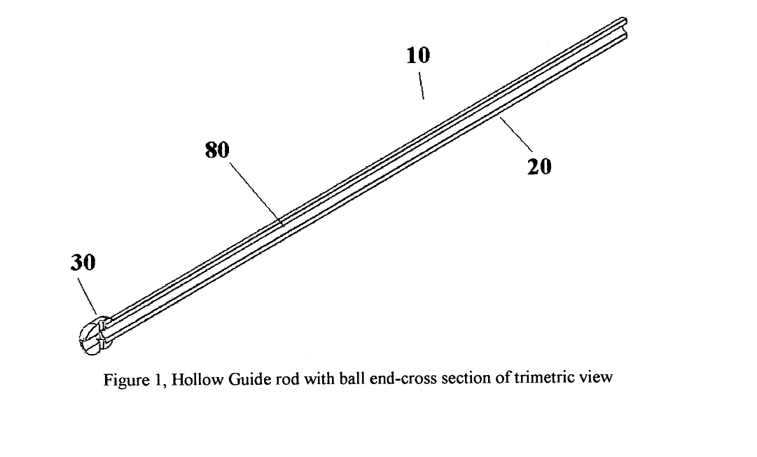

FIG. 2, Hollow Guide rod with multiple port ball end-cross section close up

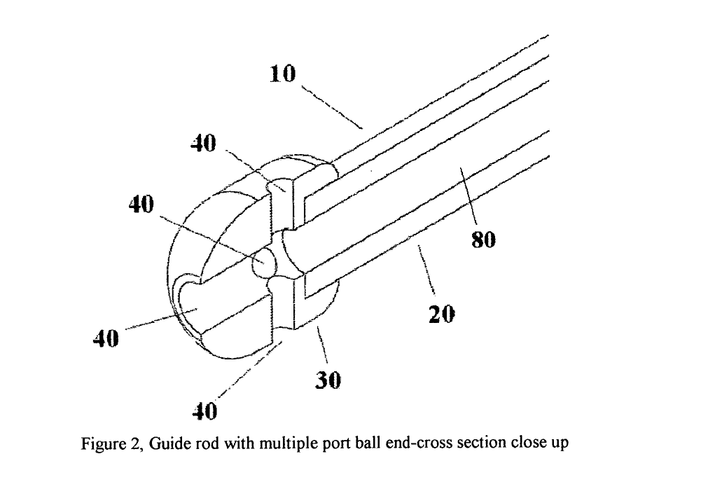

FIG. 3, Hollow Guide Rod with sensor and transmission means

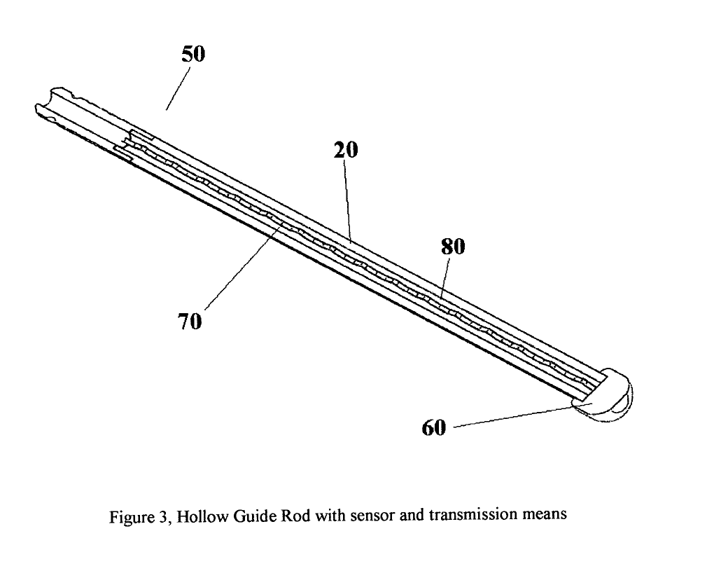

### REFERENCE NUMBER IN THE DRAWINGS

10 Hollow Guide Rod

20 Hollow tube

30 Enlarged End

40 Channels from inner to outer surface

50 Guide rod with sensor and transmission means

60 Sensor

70 Transmission means

80 Hollow Cavity

### DETAILED DESCRIPTION OF THE INVENTION

The present invention 10 is that of a conventional guide rod that is formed from a hollow tube 20. The channel 80 will allow an escape means for the pressurized fat to exit without being forced into the venous system. A small reduction in non compressible fluid volume will greatly reduce pressure.

A series of holes 40 on the end in the bone canal will reduce the likelihood that the device 10 will get plugged by debris. The tip 30 can have a plurality of holes 40 to this end also. The use of a filter, sintered or mesh will reduce the likelihood that large particles will stop the venting through the hollow cavity 80.

The surgeon could use the exiting product as a means to gage if he is advancing too fast. A slow cutting motion will have some fat coming back up, and a fast expulsion thought the guide rod end would indicate a too high canal pressure condition, indicating a need to slow the cutter advancement.

Material of the guide rod 10 will be stainless steel, titanium, cobalt, cobalt chrome or polymer.

The stiffness of a 0.125 inch diameter rod with a 0.0625 inside diameter is 93% of that of a solid rod. The polar moment of inertia for the material removed is 0.0000014980 inches to the fourth power and for the solid 0.125 inch section it is 0.0000239684 inches to the fourth power. The ratio of these is 0.9375 or rough 94% of the solid rod. For bending the difference in stiffness is the same percentage. The addition of the hollow cavity will not significantly reduce the strength of the rod needed for reduction.

The hollow channel 80 allows room for information transmission means such as electrical wires 70 that would go from a transducer or generator 60 on the tip of the rod out to a feedback means accessible by the surgeon. This transducer 60 could be added to a rod 50 that will be implanted that would be self guided and act as its own guide rod 10.

A variety of information transmission means could be transmitted from the tip 60 through the canal, electrical, ultrasonic, photonic, or magnetic. The position of the rod tip 30, would be reported back through the rod 10 to an external feedback means.

I claim:

1. A guide rod to use for long bone fracture reduction and canal preparation that has a hollow cavity along its length.

2. The device of claim 1 where the rod has an enlarged end.

3. The device of claim 1 where the guide rod has an enlarged end with a plurality of channels from inner to outer surface.

4. The device of claim 1 where the guide rod has a circular cross section.

5. The device of claim 1 where the guide rod is formed from a tube with a welded tip.

6. A method to use a hollow guide rod to add or remove substances from the bone canal during preparation for device implantation.

7. The device of claim 1 where the guide rod has a cavity containing information transmission means.

8. A guide rod for use in long bone surgery with a hollow cavity along it bore and at least one position information feedback means contained within the device.

9. The device of claim 8 where the guide rod has an integral receiver to receive signal information indicating bone and bone segment position.

10. A method to reduce a long bone fracture with a guide rod with internal signal information.
